(19) United States
(12) Patent Application Publication
(10) Pub. No.: US 2011/0224659 A1
(43) Pub. Date: Sep. 15, 2011

(54) Intrastromal Hyperplanes for Vision Correction

(76) Inventor: Luis Antonio Ruiz, Bogota (CO)

(21) Appl. No.: 13/082,155

(22) Filed: Apr. 7, 2011

Related U.S. Application Data
(63) Continuation-in-part of application No. 12/757,798, filed on Apr. 9, 2010.
Application No. 12/757,798 is a continuation-in-part of application No. 12/105,195, filed on Apr. 17, 2008, now Pat. No. 7,717,908.
Application No. 12/105,195 is a continuation-in-part of application No. 11/958,202, filed on Dec. 17, 2007, now Pat. No. 7,717,907.
(60) Provisional application No. 61/322,718, filed on Apr. 9, 2010.

(51) Int. Cl. A61F 9/008

(52) U.S. Cl. 606/5

(57) ABSTRACT

A system and method for influencing the asphericity of the cornea of an eye requires creating a cut inside the stroma by Laser Induced Optical Breakdown (LIOB). Specifically, this cut is made over a substantially hyperbolic surface that is substantially centered on the visual axis of the eye, with its curvature opposite the curvature of the cornea. The cut can be made separately, or in conjunction with other LIOB cuts that are introduced to correct specific vision defects.

This application claims the benefit of U.S. Provisional Patent Application Ser. No. 61/322,718, filed Apr. 9, 2010 and is a continuation-in-part of application Ser. No. 12/757,798 filed Apr. 9, 2010, which is currently pending, which is a continuation-in-part of U.S. Pat. No. 7,717,908, which is a continuation-in-part of U.S. Pat. No. 7,717,907. The contents of application Ser. No. 12/757,798, and U.S. Pat. Nos. 7,717,908 and 7,717,907, are incorporated herein by reference.

### FIELD OF THE INVENTION

The present invention pertains generally to systems and methods for performing Laser Induced Optical Breakdown (LIOB) in stromal tissue to provide refractive corrections for an eye. More particularly, the present invention pertains to systems and methods wherein LIOB is performed over an aspheric surface inside the stroma of an eye. The present invention is particularly, but not exclusively, useful for creating a substantially hyperbolic cut inside the stroma of a cornea wherein the curvature of the cut is opposite the curvature of the cornea.

### BACKGROUND OF THE INVENTION

As is well known, there are many factors that contribute to a person's visual acuity. It happens that one of these factors involves the asphericity of the cornea of an eye (i.e. the corneal Q value). As a surgical consideration, changing this so-called “Q” value may, in certain instances, be of help in improving the person's vision.

Structurally, the cornea of an eye is continually influenced by both external and internal forces. In a direction along the visual axis, these forces include the external force that is created on the anterior surface of the eye by the atmosphere. This force is opposed by another force, also external to the cornea, that is imposed on the posterior surface of the cornea by intraocular pressure (IOP) inside the eye. These external forces, however, are not equal to each other and consequently they impose a pressure differential on the cornea that is balanced by internal biomechanical forces that are generated by corneal tissue inside the cornea. Some of these forces are generated inside the stroma of the cornea.

It has been shown that by weakening stromal tissue, such as with LIOB cuts, the cornea will respond to the pressure differential that is created by the external forces. In particular, it is known that such a response results in the cornea changing its shape. An intended consequence here has been to create these cuts in order to reshape the cornea for the correction of vision defects such as presbyopia, hyperopia and myopia. As implied above, in addition to the correction of these known defects, the asphericity (i.e. “Q” factor) of a cornea can have an overarching effect that may need to be considered for correction either separately by itself, or together with other defects.

In light of the above it is an object of the present invention to provide a system and method for correcting corneal asphericity by weakening the cornea of an eye with LIOB cuts. Another object is to provide a system and method for cutting stromal tissue on a defined surface(s) inside the stroma to influence the asphericity of the cornea. Still another object of the present invention is to provide a system and method for correcting corneal asphericity that is easy to use, simple to manufacture and cost effective.

### SUMMARY OF THE INVENTION

A system and method are provided for photo-altering an aspheric stratum of transparent flexible material, such as the stroma in a cornea of an eye. For the present invention, this photo-alteration requires Laser Induced Optical Breakdown (LIOB) of the material over at least a portion of a defined surface within the stratum (stroma). This LIOB is accomplished using a laser unit that generates laser pulses. Specifically, each laser pulse will have a pulse duration that is less than one picosecond, and a pulse energy in a range of approximately 10 nJ to approximately 1 μJ. For purposes of the present invention, the defined surface will be curved, with one side of the defined surface being substantially concave. More specifically, the concave side of the defined surface can be substantially spherical, parabolic, hyperbolic or elliptical. In the context of a cornea (stroma) the concave side of the defined surface will be its anterior side.

As envisioned for the present invention, the methodology includes first determining a thickness (T) for the stratum (stroma). In this case, T is the distance between an anterior surface and a posterior surface of the stratum. Next, an axis is identified which is substantially perpendicular to the anterior surface of the stratum (e.g. the visual axis of an eye). The defined surface inside the stratum (stroma) can then be mathematically defined by varying a radius vector (R). In detail, the origin of the radius vector will lie on the axis and will be anterior to the defined surface. Further, the radius vector has a variable length (l) that is measured from the origin; it has a variable rotation angle (θ) that is measured around the axis; and it has an inclination angle (φ) that is measured between the axis and the radius vector, relative to the axis. As mentioned above, the anterior side of the defined surface is substantially concave.

Once a defined surface has been identified and defined, a laser surgical procedure can be performed on the surface. Specifically, this is done by moving the focal point of a laser beam from point to point on the defined surface to create LIOB at a plurality of points. Not all points on the surface, however, need to be photo-altered by LIOB.

In order to appreciate the many possible configurations for a defined surface, to also geometrically define the collective limitations for such a surface, and to accurately locate areas of the surface for LIOB, it is helpful to establish several geometric definition points for the defined surface. With these objects in mind, it is to be appreciated that the defined surface will be centered on the axis. Also, the defined surface will have a substantially circular periphery and, as mentioned above, it will be concave.

As a start point for describing the defined surface, and for discussing various LIOB possibilities, a base point (p_{base}) is located at the intersection of the axis with the defined surface. Preferably, p_{base} is posterior to the origin of the radius vector. Separate from the base point, p_{base}, an end point (p_{end}) can be established on the periphery of the defined surface at a maximum inclination angle (φₘₐₓ). A distance between p_{end} at a rotation angle θ, and p_{end} at a rotation angle θ+180°, will then define a diameter (D) for the defined surface. Collectively, the p_{end} locations in the stratum for a rotation of the radius vector through a θof 360° define the periphery of the defined surface.

For purposes of the present invention, several geometric limitations for the defined surface are particularly important. For one, φₘₐₓ must always be established to maintain each p_{end} at a location within the stratum (stroma). To do this, every p_{end} must be posterior to the anterior surface of the stratum. Another important geometrical limitation for the defined surface is that p_{base} is preferably located at a distance less than approximately 0.8T from the anterior surface of the stratum (stroma). And, D is typically established to be in a range between 4 mm and 7 mm.

Insofar as areas on the defined surface where LIOB is to be performed are concerned, the present invention envisions several possibilities. For one, areas for LIOB can be identified by moving R through rotations of Δθ, wherein Δθ is less than 360°. Further, axially opposed rotations of Δθ are also envisioned wherein each rotation Δθ is less than 180°. Also, as another variation, R can be moved through rotation of Δφ. For example, Δφ may extend anywhere between a minimum inclination angle φₘᵢₙ and φₘₐₓ, wherein φₘᵢₙ is greater than zero. Further, several different defined surfaces can be used for a same surgical procedure. If so, each defined surface will have its own unique base point p_{base}. In yet another variation for the methodology of the present invention, and in addition to the defined surfaces, LIOB can also be performed on cylindrical surfaces. If incorporated, each cylindrical surface will have a diameter “d”, wherein d is less than D. Further, the cylindrical surface will be centered on the axis, and will be completely located within the stratum.

Structurally, a system for implementing the methodologies of the present invention includes a programmable computer and a laser unit for generating the laser beam. Within this structure, the laser unit is controlled and operated by the computer in accordance with the computer program. More specifically, the computer program varies the radius vector (R) to mathematically define the defined surface inside the stratum. As noted above, the origin of the radius vector lies on the axis and it is preferably anterior to the defined surface. Further, the computer program controls the laser unit to move the focal point of the laser beam from point to point on the defined surface to create LIOB at a plurality of points on the defined surface to weaken the stratum and influence its aspheric condition.

### BRIEF DESCRIPTION OF THE DRAWINGS

FIG. 1 is a cross sectional view of the cornea of a patient depicting various stromal surfaces on which LIOB may be accomplished in accordance with the present invention; and

FIG. 2 is a view of the cornea shown in FIG. 1, in conjunction with a laser unit for LIOB, wherein the geometric measurements for defining surfaces for LIOB, and for establishing their geometric limitations are presented.

### DESCRIPTION OF THE PREFERRED EMBODIMENTS

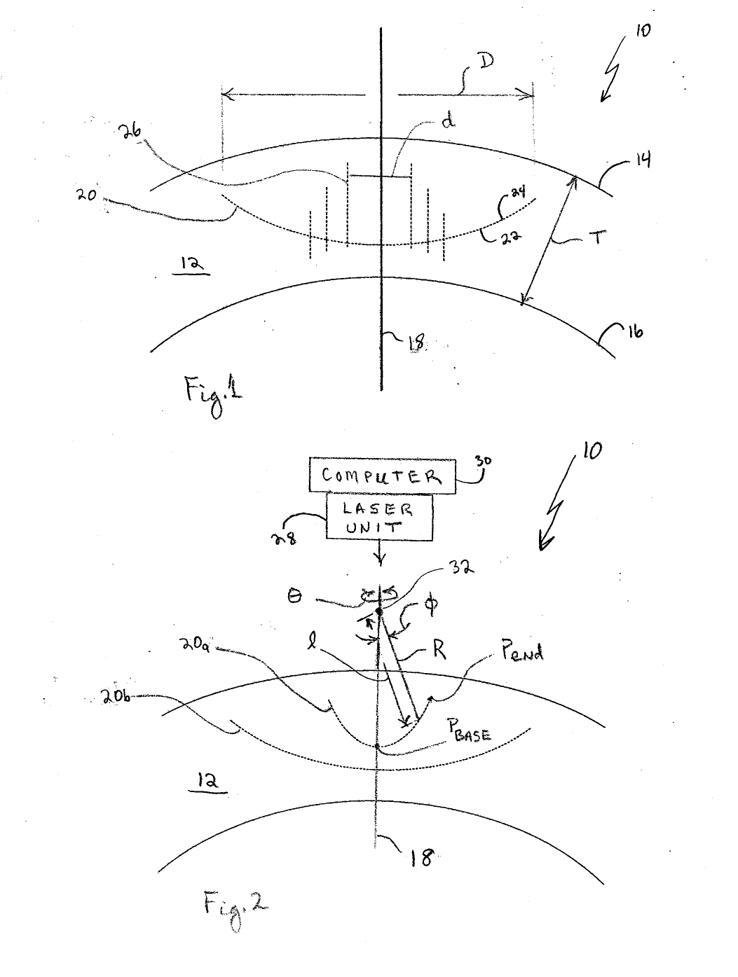

Referring initially to FIG. 1, a cross sectional view of a portion of a cornea is shown and generally designated 10. In particular, a stratum 12 of the cornea 10 is shown, with the stratum 12 being the area within the cornea 10 where a laser surgical procedure will occur in accordance with the present invention. As shown, the stratum 12 has a thickness “T” and is further defined by an anterior surface 14 and a posterior surface 16. Additionally, an axis 18 is identified which is substantially perpendicular to the anterior surface 14 of the stratum 12. This axis 18 will typically be the visual axis of an eye. As required for the present invention, a defined surface 20 having a diameter “D” is defined and identified within the stratum 12. In detail, the defined surface 20 is the area where Laser Induced Optical Breakdown (LIOB) is performed in accordance with the present invention. In further detail, “D” is the straight-line distance between the two ends of the defined surface 20 and is envisioned to be between 4 mm and 7 mm. As can be seen, the defined surface 20 is curved with a concave side 24 facing the anterior surface 14 and a convex side 22 facing the posterior surface 16. As shown in FIG. 1, the defined surface 20 is generally parabolic in shape in a preferred embodiment. In other embodiments, the defined surface 20 may also be spherical, hyperbolic, or elliptical in shape. Alternatively, or additionally, a cylindrical surface 26 having a diameter “d” may be selected for LIOB. As shown, the cylindrical surface 26 is wholly contained within the stratum 12 of the cornea 10 like the defined surface 20. Like the diameter “D” for the defined surface 20, the diameter “d” also is the straight-line distance between opposing edges of the cylindrical surface 26. In the case of either the defined surface 20 or the cylindrical surface 26, photo-alteration does not necessarily have to occur on all points of either surface.

Now referring to FIG. 2, structural and geometric details required for the present invention are described and illustrated. Structurally, a laser unit 28 for producing a laser beam and a computer 30 are provided to implement the method of the present invention. More specifically, the computer 30 is programmed to provide instructions to the laser unit 28. Once the laser unit 28 receives the instructions, photo-alteration (LIOB) is carried out in the stratum 12 of the cornea 10. Specifically, and as described previously, LIOB is performed on a defined surface 20 or a cylindrical surface 26 by moving a focal point of the laser beam from point to point on the defined surface 20. As shown in FIG. 2, more than one defined surface 20a, 20b may be designated for use with the present invention.

Still referring to FIG. 2, it can be seen that the geometric details required for identifying the defined surface 20a, 20b for the present invention are defined relative to the axis 18. It should be noted that the geometric details used to delineate defined surface 20a may also be used to delineate defined surface 20b. As such, geometric details discussed relative to defined surface 20a may also be used with respect to defined surface 20b. Mathematically, the defined surface 20a is identified by a radius vector “R.” This radius vector “R” has an origin 32 which lies on the axis 18 and is located outside the stratum 12. In detail, the radius vector “R” has a variable length “l” and a variable rotation angle “θ” that are both measured from the origin 32.

Furthermore, the radius vector “R” also has an inclination angle (φ) that is measured relative to the axis 18 between the axis 18 and the radius vector “R”.

Again referring to FIG. 2, the defined surface 20a can be identified with specificity by using the geometric details described above. Initially, to identify the defined surface 20a, a base point (p_{base}) identified at the intersection of the defined surface 20a with the axis 18. In a preferred embodiment, p_{base} is located approximately 0.8T from the anterior surface 14 of the stratum 12. As shown, p_{base} is located posterior to the origin 32 of the radius vector “R.” The defined surface 20a also has two end points (p_{end}) established on the periphery of the defined surface 20a at a maximum inclination angle (φₘₐₓ). The diameter “D” can be defined for the defined surface 20a as the distance between p_{end} at a rotation angle θ and p_{end} at a rotation angle θ+180°. When taken collectively, the p_{end} locations in the stratum 12, for a rotation of the radius vector “R” through a rotation angle θ of 360°, define the periphery of the defined surface 20a. In addition, to ensure the defined surface 20a is located within the stratum 12, φₘₐₓ must always be established so every p_{end} remains posterior to the anterior surface 14 of the stratum 12.

Using the geometric variables shown in FIG. 2, several embodiments of the present invention can be described. In one embodiment, the defined surface 20a can be identified by moving radios vector “R” through rotations of Δθ with θ being less than 360°. In another embodiment, axially opposed rotations of Δθ may be used as long as each rotation is less than 180°. In yet another embodiment, radius vector “R” can be moved through a rotation of Δθ, with Δ74 extending anywhere between a minimum inclination angle (φₘᵢₙ) and a maximum inclination angle of (φₘₐₓ). In this embodiment, the value for (φₘᵢₙ) must be greater than zero.

While the particular Intrastromal Hyperplanes for Vision Correction as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A method for creating Laser Induced Optical Breakdown (LIOB) over a defined surface inside an aspheric stratum of transparent flexible material, to influence the aspheric condition of the stratum, wherein the method comprises the steps of:
determining a thickness (T) for the stratum, wherein T is a distance between an anterior surface and a posterior surface of the stratum;
identifying an axis, wherein the axis is substantially perpendicular to the anterior surface of the stratum;
varying a radius vector (R) to mathematically define the defined surface inside the stratum, wherein the origin of the radius vector lies on the axis and is anterior to the defined surface, and further wherein the anterior side of the defined surface is substantially concave; and
moving a focal point of a laser beam from point to point on the defined surface to create LIOB at a plurality of points on the defined surface to weaken the stratum and influence its aspheric condition.

2. A method as recited in claim 1 wherein the radius vector has a variable length (l) measured from its origin, a variable rotation angle (θ) measured around the axis, and an inclination angle (φ) measured relative to the axis.

3. A method as recited in claim 2 further comprising the steps of:
locating a base point (p_{base}) at an intersection of the axis with the defined surface, wherein p_{base} is posterior to the origin of the radius vector; and
establishing an end point (p_{end}) on a periphery of the defined surface at a maximum inclination angle (φₘₐₓ), wherein a distance between p_{end} at θ and p_{end} at 74 +180° define an end point diameter (D) for the defined surface.

4. A method as recited in claim 3 wherein φₘₐₓ is established to maintain p_{end} posterior to the anterior surface of the stratum.

5. A method as recited in claim 3 wherein p_{base} is located at a distance less than approximately 0.8T from the anterior surface of the stratum.

6. A method as recited in claim 3 wherein D is established to be in a range between 4 mm and 7 mm.

7. A method as recited in claim 3 wherein R is moved through axially opposed rotations of Δθ, wherein each rotation Δθ is less than 180°, to establish sectors of LIOB.

8. A method as recited in claim 3 wherein R is moved between a minimum inclination angle φₘᵢₙ and φₘₐₓ, and wherein φₘᵢₙ is greater than zero.

9. A method as recited in claim 3 wherein there is a plurality of defined surfaces with each defined surface having a unique base point p_{base}.

10. A method as recited in claim 3 further comprising the step of creating at least one cylindrical surface for LIOB wherein the cylindrical surface has a diameter “d”, is centered on the axis, is located within the stratum, and further wherein d is less than D.

11. A method as recited in claim 3 wherein the defined surface is substantially spherical.

12. A method as recited in claim 3 wherein the defined surface is substantially parabolic.

13. A method as recited in claim 3 wherein the defined surface is substantially hyperbolic.

14. A method as recited in claim 3 wherein LIOB is accomplished with laser pulses, wherein each laser pulse has a pulse duration less than one picosecond.

15. A method as recited in claim 14 wherein each pulse has a pulse energy in a range of approximately 10 nJ to approximately 1 μJ.

16. A system for creating Laser Induced Optical Breakdown (LIOB) over a defined surface inside an aspheric stratum of transparent flexible material to influence the aspheric condition of the stratum, wherein the stratum defines an axis and the system comprises:
a laser unit for generating a laser beam; and
a computer for controlling the laser beam in accordance with a computer program to vary a radius vector (R) to mathematically define the defined surface inside the stratum, wherein the origin of the radius vector lies on the axis and is anterior to the defined surface, and wherein the computer program controls the laser unit to move a focal point of the laser beam from point to point on the defined surface to create LIOB at a plurality of points on the defined surface to weaken the stratum and influence its aspheric condition.

17. A system as recited in claim 16 wherein the radius vector has a variable length (l) measured from its origin, a variable rotation angle (θ) measured around the axis, and an inclination angle (φ) measured relative to the axis.

18. A system as recited in claim 17 wherein R is moved through axially opposed rotations of Δθ and between a minimum inclination angle φₘᵢₙ and a maximum inclination angle φₘₐₓ.

19. A system as recited in claim 16 wherein LIOB is accomplished with laser pulses, wherein each laser pulse has a pulse duration less than one picosecond.

20. A system as recited in claim 19 wherein each pulse has a pulse energy in a range of approximately 10 nJ to approximately 1 μJ.
